(19) United States
(12) Patent Application Publication
(10) Pub. No.: US 2025/0372207 A1
(43) Pub. Date: Dec. 4, 2025

(54) METHOD AND SYSTEM FOR COMPREHENSIVE WATER QUALITY ASSESSMENT BY INTEGRATING BIOTIC AND ABIOTIC FACTORS

(71) Applicant: Peking University, Beijing (CN)

(72) Inventors: Qian CHEN, Beijing (CN); Tianyi CHEN, Beijing (CN); Weiling SUN, Beijing (CN); Jinren NI, Beijing (CN)

(21) Appl. No.: 18/826,277

(22) Filed: Sep. 6, 2024

(30) Foreign Application Priority Data
May 28, 2024 (CN) 202410666328.0

(51) Int. Cl. G16B 35/00; G01N 33/18; G16B 40/30; G16C 20/70

(52) U.S. Cl. CPC G16B 35/00; G01N 33/18; G16B 40/30; G16C 20/70

(57) ABSTRACT

Provided is a method and system for comprehensive water quality assessment by integrating biotic and abiotic factors. The method includes: acquiring abiotic factors of a water body to be tested; constructing a biotic factor indicator library by an environmental DNA technology; determining a biotic-abiotic response relationship-based abiotic factor weight matrix using the abiotic factors and the biotic factor indicator library; acquiring a machine learning-based abiotic factor weight matrix using the abiotic factors and a LightGBM model; determining an abiotic factor comprehensive weight matrix according to the biotic-abiotic response relationship-based abiotic factor weight matrix and the machine learning-based abiotic factor weight matrix; and conducting the comprehensive water quality assessment of the water body to be tested based on the abiotic factor comprehensive weight matrix and the abiotic factors to determine a comprehensive water quality assessment result of the water body to be tested.

### CROSS REFERENCE TO RELATED APPLICATION

This patent application claims the benefit and priority of Chinese Patent Application No. 2024106663280, filed with the China National Intellectual Property Administration on May 28, 2024, the disclosure of which is incorporated by reference herein in its entirety as part of the present application.

### TECHNICAL FIELD

The present disclosure relates to the technical field of environmental monitoring and environmental protection, and in particular to a method and system for comprehensive water quality assessment by integrating biotic and abiotic factors.

### BACKGROUND

The water quality assessment for rivers, lakes, and reservoirs refers to the selection of corresponding assessment criteria, parameters, and methods according to the use and function of target water to assess a quality of the water. In recent years, the rapid population growth and the surge in the consumption of industrial and agricultural water have caused the continuous deterioration of water qualities of aquatic ecosystems of rivers and lakes, posing a huge risk to the global water safety and ecological management. The establishment of a reliable and effective water quality assessment method to accurately and rapidly measure a water quality of a natural aquatic ecosystem is a pressing challenge faced by government managers and environmental scholars. Currently, surface water quality assessment systems are widely established based on physical and chemical water quality parameters, such as the typical water quality index (WQI) method. In the WQI method, a score of 0 to 100 is assigned to a quality of water, and then whether the water can be used as drinking water, irrigation water, landscape water, or the like is determined according to the score.

The establishment of a water quality assessment system generally includes the following three aspects: selection of assessment indicators, determination of an assessment method, and assignment of indicator weights. Although the research methods and theoretical systems for comprehensive water quality assessment have been developed successively with the increasing attention to water resource management and water supply safety, there are still the following problems at an operational level. In terms of the selection of assessment indicators, a complete system is established based on indicators such as conventional physical and chemical properties and inorganic substances. In addition to conventional indicators, certain emerging contaminants closely linked to human activities can also pose toxic risks to aquatic organisms. However, current water quality assessment systems often overlook these emerging contaminants and lack a comprehensive framework that systematically incorporates diverse abiotic factors in water for assessment purposes. In addition, there are many uncertainties in an aquatic environment itself, and both the classification of a water quality grade and the establishment of aquatic environment quality standards are ambiguous. In the existing water quality safety assessment systems, the subjective analysis and determination dominate in terms of the assignment of indicator weights. Although there are methods such as fuzzy comprehensive assessment and artificial neural network models to reduce the influence of subjective analysis and determination, the objectivity and accuracy of an assessment result still need to be improved.

In fact, in addition to abiotic factors, water ecosystems include biotic factors across multiple trophic levels, including algae, bacteria, fungi, archaea, zoobenthos, and fish. The European Water Framework Directive (WFD) proposes that the establishment of environmental quality standards should take into account both physical and chemical factors (such as nutrient concentration, pH, and suspended solid concentration) and biotic quality factors (such as biodiversity, food web integrity, and community stability). On the one hand, the structure and function of biotic communities are extremely sensitive to changes in environmental conditions, and can comprehensively and quickly reflect ecological process changes caused by variations in abiotic factors in water. On the other hand, with the rapid development of modern molecular biology and environmental DNA technology, the composition and functional diversity of biological communities can be rapidly detected to systematically characterize the structural and functional integrity of an ecosystem. However, researchers have not yet developed a fully mature method and theory for integrating biotic community and functional information into a water quality safety assessment system.

### SUMMARY

An objective of the present disclosure is to provide a method and system for comprehensive water quality assessment by integrating biotic and abiotic factors, which can comprehensively, accurately, and quickly allow the multivariate comprehensive water quality assessment.

To allow the above objective, the present disclosure provides the following solutions:

A method for comprehensive water quality assessment by integrating biotic and abiotic factors is provided, including the following steps:

acquiring abiotic factors of a water body to be tested, where the water body to be tested includes a river, a lake, and a reservoir; the abiotic factors include different abiotic indicators; and the different abiotic indicators are pH, dissolved oxygen, total dissolved solids, a permanganate index, ammonia nitrogen, nitrate nitrogen, total nitrogen, total phosphorus (TP), chlorides, sulfates, Na, Fe, Ca, Mg, Cu, Zn, Cr, As, Mo, antibiotics, or perfluorinated compounds;
constructing a biotic factor indicator library by an environmental DNA technology, where the biotic factor indicator library includes different biotic indicators of biotic communities at different trophic levels; the biotic communities at different trophic levels are bacterial communities, archaeal communities, fungal communities, algal communities, zoobenthic communities, or fish communities; and the different biotic indicators are diversity indexes, relative abundances at each classification level, or co-occurrence network topology properties;
determining a biotic-abiotic response relationship-based abiotic factor weight matrix using the abiotic factors and the biotic factor indicator library;
acquiring a machine learning-based abiotic factor weight matrix using the abiotic factors and a LightGBM model, where the LightGBM model is configured to determine importance of each abiotic indicator among the abiotic factors relative to water quality;
determining an abiotic factor comprehensive weight matrix according to the biotic-abiotic response relationship-based abiotic factor weight matrix and the machine learning-based abiotic factor weight matrix; and
conducting the comprehensive water quality assessment of the water body to be tested based on the abiotic factor comprehensive weight matrix and the abiotic factors to determine a comprehensive water quality assessment result of the water body to be tested, where the comprehensive water quality assessment result is provided to characterize a water quality safety status.

A computer system is provided, including: a memory, a processor, and a computer program stored in the memory and runnable on the processor, where the processor is configured to execute the computer program to implement the steps of the method for comprehensive water quality assessment by integrating biotic and abiotic factors described above.

According to the specific embodiments provided by the present disclosure, the present disclosure discloses the following technical effects: The present disclosure discloses a method and system for comprehensive water quality assessment by integrating biotic and abiotic factors. The method includes: acquiring abiotic factors of a water body to be tested; constructing a biotic factor indicator library by an environmental DNA technology; determining a biotic-abiotic response relationship-based abiotic factor weight matrix using the abiotic factors and the biotic factor indicator library; acquiring a machine learning-based abiotic factor weight matrix using the abiotic factors and a LightGBM model; determining an abiotic factor comprehensive weight matrix according to the biotic-abiotic response relationship-based abiotic factor weight matrix and the machine learning-based abiotic factor weight matrix; and conducting the comprehensive water quality assessment of the water body to be tested based on the abiotic factor comprehensive weight matrix and the abiotic factors to determine a comprehensive water quality assessment result of the water body to be tested, where the comprehensive water quality assessment result is provided to characterize a water quality safety status. The present disclosure can comprehensively, accurately, and quickly allow the comprehensive water quality assessment.

### BRIEF DESCRIPTION OF THE DRAWINGS

To describe the technical solutions in the embodiments of the present disclosure or in the prior art clearly, the accompanying drawings required for the embodiments are briefly described below. Apparently, the accompanying drawings in the following description show merely some embodiments of the present disclosure, and those of ordinary skill in the art may still derive other accompanying drawings from these accompanying drawings without creative efforts.

FIG. 1 is a schematic flow chart of the method for comprehensive water quality assessment by integrating biotic and abiotic factors provided by the present disclosure;

FIG. 2 shows a 0-1 correlation matrix;

FIG. 3 shows a WQI value variation and a water quality grade composition along a river;

FIG. 4 shows a fitting trend chart of a biotic-abiotic response relationship-based abiotic factor weight matrix W_{mic}

FIG. 5 shows a fitting trend chart of a machine learning-based abiotic factor weight matrix W_{LGBM}, and

FIG. 6 shows a fitting trend chart of an abiotic factor comprehensive weight matrix W.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the embodiments are merely some rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide a method and system for comprehensive water quality assessment by integrating biotic and abiotic factors, which can comprehensively, accurately, and quickly allow the comprehensive water quality assessment.

Based on the interdependence and interaction between different biotic factors and abiotic factors in river and lake (reservoir) systems, the present disclosure inventively proposes to calculate and characterize an indicator weight based on a biotic-abiotic factor response relationship and a machine learning model, such that the relative importance information of an indicator that is reasonable, scientific, and practical according to actual tests can be obtained, which ensures the objectivity and practicability of the indicator weight.

The method of the present disclosure includes the following steps: monitoring of abiotic factors; monitoring of biotic factors; construction of a biotic factor indicator library; calculation of a biotic-abiotic response relationship-based abiotic factor weight matrix; calculation of a machine learning-based abiotic factor weight matrix; calculation of a water quality assessment index; and output of an assessment result. In the present disclosure, a plurality of abiotic and biotic factors are monitored, an indicator weight is quantified through a biotic and abiotic response relationship and a machine learning model, and a comprehensive assessment index is constructed to comprehensively assess the water quality safety of rivers, lakes, and reservoirs, which can effectively avoid the one-sidedness of assessment results due to limited assessment indicators, is conducive to avoiding the uncertainty caused by subjective determination, and provides a technical support for the multivariate comprehensive water quality assessment of rivers, lakes, and reservoirs.

In order to make the above objective, features, and advantages of the present disclosure clear and comprehensible, the present disclosure will be further described in detail below in combination with the accompanying drawings and specific implementations.

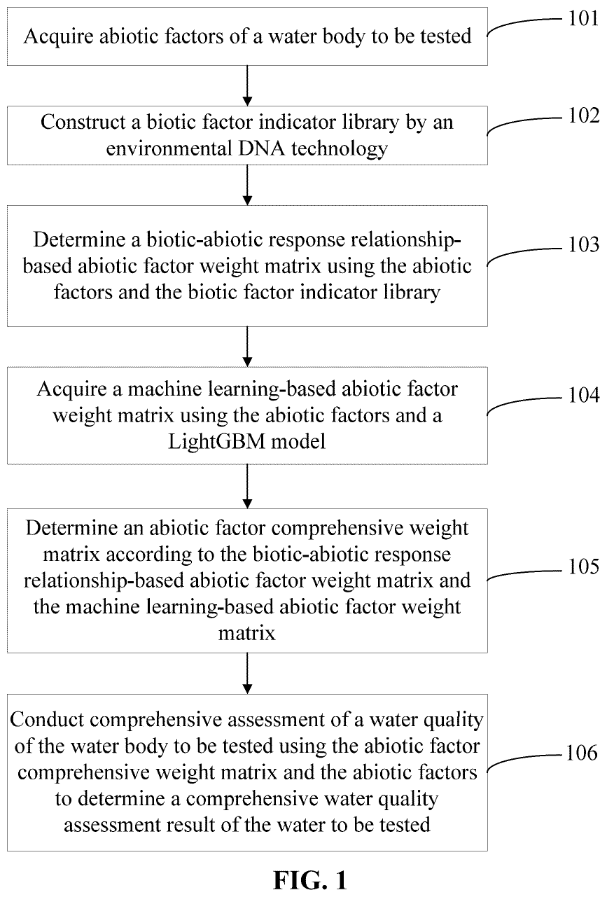

Example 1: As shown in FIG. 1, a method for comprehensive water quality assessment by integrating biotic and abiotic factors is provided in this example, including the following steps:

In accordance with principles in a standard/specification, sampling sites are set and water samples are collected for monitoring of abiotic factors, including the determination of basic physical and chemical properties such as pH, dissolved oxygen, total dissolved solids, a permanganate index, ammonia nitrogen, nitrate nitrogen, total nitrogen, TP, chlorides, and sulfates and the determination of concentrations of heavy metals such as Na, Fe, Ca, Mg, Cu, Zn, Cr, As, and Mo and emerging contaminants such as antibiotics and perfluorinated compounds.

Step 101: Abiotic factors of a water body to be tested are acquired. The water body to be tested includes a river, a lake, and a reservoir; the abiotic factors include different abiotic indicators; and the different abiotic indicators can be pH, dissolved oxygen, total dissolved solids, a permanganate index, ammonia nitrogen, nitrate nitrogen, total nitrogen, TP, chlorides, sulfates, Na, Fe, Ca, Mg, Cu, Zn, Cr, As, Mo, antibiotics, or perfluorinated compounds.

A barcode fragment is amplified with the acquired environmental DNA as a template for biotic communities across multiple trophic levels such as bacteria, fungi, archaea, algae, zoobenthos, and fish, high-throughput sequencing is conducted, and a relative abundance and a species annotation of a corresponding operational taxonomic unit (OTU) at a sampling point are determined based on the acquired high-throughput sequencing data.

The Alpha diversity indexes such as ACE, Chao, Shannon, and Simpson indexes of biotic communities at different trophic levels are calculated. Relative abundances of bacterial, archaeal, fungal, algal, zoobenthic, and fish communities are calculated at each classification level. A co-existence relationship network of bacterial, archaeal, fungal, algal, zoobenthic, and fish communities is constructed. Co-occurrence network topology properties such as a node number, an edge number, a network degree, assortativity, an edge density, an average path length, betweenness centrality, degree centralization, network transitivity, a network diameter, modularity, and vulnerability are calculated.

Step 102: A biotic factor indicator library is constructed by the environmental DNA technology. The biotic factor indicator library includes different biotic indicators of biotic communities at different trophic levels. The biotic communities at different trophic levels are bacterial communities, archaeal communities, fungal communities, algal communities, zoobenthic communities, or fish communities. The different biotic indicators are diversity indexes, relative abundances at each classification level, or co-occurrence network topology properties.

High-throughput sequencing data of the biotic communities at different trophic levels in the water body to be tested is acquired by the environmental DNA technology.

The high-throughput sequencing data of the biotic communities at different trophic levels is subjected to quality control and filtration to obtain processed high-throughput sequencing data of the biotic communities at different trophic levels.

The processed high-throughput sequencing data of the biotic communities at different trophic levels is clustered to obtain OTU representative sequences.

The OTU representative sequences are subjected to taxonomic annotation by a taxonomic approach to calculate diversity indexes, relative abundances at each classification level, or co-occurrence network topology properties of the biotic communities at different trophic levels. The diversity indexes include ACE, Chao, Shannon, and Simpson indexes. The co-occurrence network topology properties include at least one of a node number, an edge number, a network degree, assortativity, an edge density, an average path length, betweenness centrality, degree centralization, network transitivity, a network diameter, modularity, and vulnerability.

The taxonomic approach is any one of a ribosomal database project (RDP) classifier Bayesian algorithm and a basic local alignment search tool (BLAST) alignment approach.

Step 103: A biotic-abiotic response relationship-based abiotic factor weight matrix is determined using the abiotic factors and the biotic factor indicator library.

Spearman correlation between each abiotic indicator among the abiotic factors and each biotic indicator of the biotic communities at different trophic levels in the biotic factor indicator library is calculated.

The Spearman correlation between each abiotic indicator among the abiotic factors and each biotic indicator of the biotic communities at different trophic levels in the biotic factor indicator library is tested to obtain a significance P value between each abiotic indicator among the abiotic factors and each biotic indicator of the biotic communities at different trophic levels in the biotic factor indicator library.

A significance P value matrix is constructed based on the significance P value between each abiotic indicator among the abiotic factors and each biotic indicator of the biotic communities at different trophic levels in the biotic factor indicator library.

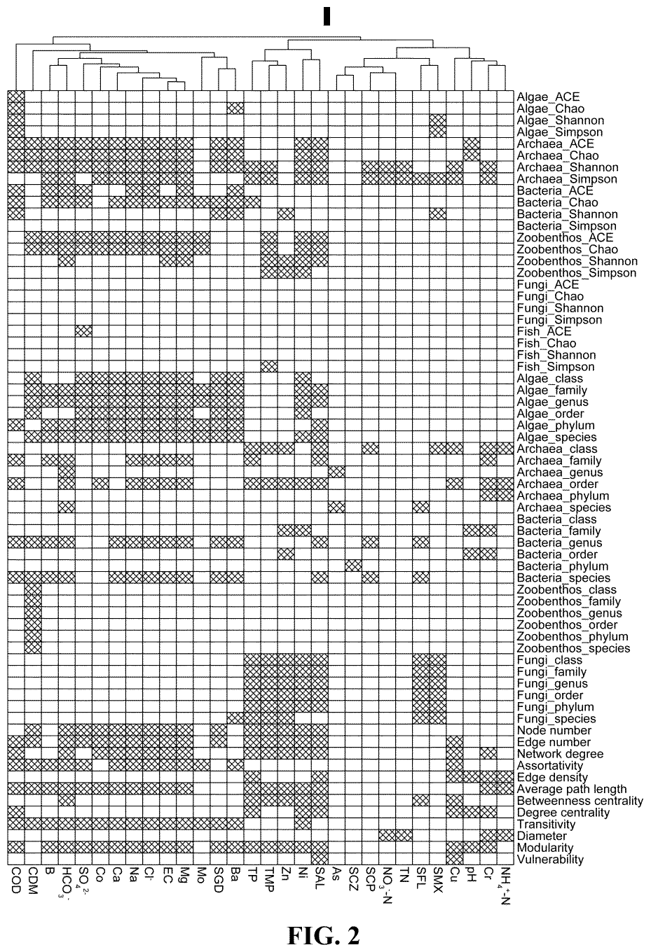

A significance P value in the significance P value matrix that satisfies a preset condition is defined as 1, and a significance P value in the significance P value matrix that does not satisfy the preset condition is defined as 0, so as to obtain a 0-1 correlation matrix, as shown in FIG. 2.

The preset condition is as follows: When the significance P value between each abiotic indicator among the abiotic factors and each biotic indicator of the biotic communities at different trophic levels in the biotic factor indicator library is smaller than 0.05, it indicates that there is a correlation, and the significance P value is defined as 1. When the significance P value between each abiotic indicator among the abiotic factors and each biotic indicator of the biotic communities at different trophic levels in the biotic factor indicator library is larger than 0.05, it indicates that there is no correlation, and the significance P value is defined as 0.

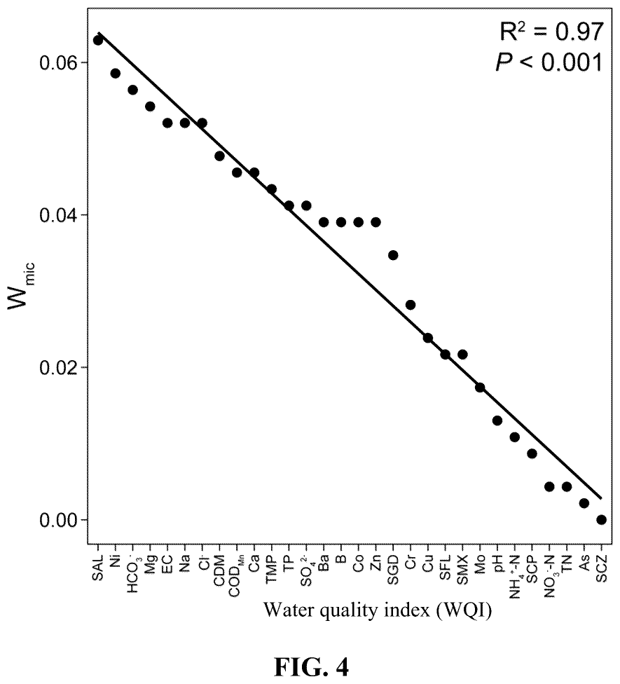

The 0-1 correlation matrix is standardized and normalized to obtain the biotic-abiotic response relationship-based abiotic factor weight matrix W_{mic}, as shown in FIG. 4.

$${C}_{i} = \frac{{n}_{i}}{N} , \\ {C}_{1} ( scale ) = \frac{{C}_{i} - min ( [ {C}_{1} , … {C}_{n} ] )}{max ( [ {C}_{1} , … {C}_{n} ] ) - min ( [ {C}_{1} , … {C}_{n} ] )} , \\ and \\ {W}_{mic} = [ \begin{matrix}\frac{{C}_{1} ( scale )}{{\sum }_{i = 1}^{n} {C}_{i} ( scale )} \\ \frac{{C}_{2} ( scale )}{{\sum }_{i = 1}^{n} {C}_{i} ( scale )} \\ ⋮ \\ \frac{{C}_{n} ( scale )}{{\sum }_{i = 1}^{n} {C}_{i} ( scale )}\end{matrix} ]$$

where N represents a number of abiotic indicators among the abiotic factors, and the abiotic indicators among the abiotic factors are ranked from high to low in terms of importance; Cᵢ represents a Spearman correlation degree of an ith abiotic indicator among the abiotic factors; Cₙ represents a Spearman correlation degree of an nth abiotic indicator among the abiotic factors; nᵢ represents a total number of biotic indicators that are significantly correlated to the i th abiotic indicator among the abiotic factors; Cᵢ (scale) represents a Spearman correlation degree of the ith abiotic indicator among the abiotic factors after standardization; Cₙ(scale) represents a Spearman correlation degree of the nth abiotic indicator among the abiotic factors after standardization; max represents a maximum value; and min represents a minimum value.

Step 104: A machine learning-based abiotic factor weight matrix is acquired using the abiotic factors and a LightGBM model. The LightGBM model is configured to determine importance of each abiotic indicator among the abiotic factors relative to water quality.

According to the Environmental Quality Standards for Surface Water (GB3838-2002) and relevant standards/specifications such as emerging contaminant toxicity, a water quality category at each sampling site (subject to the worst indicator category) is determined, and the importance ranking of each abiotic indicator among the abiotic factors is determined with the LightGBM machine learning algorithm. The LightGBM model is based on a gradient boosting decision tree (GBDT) model optimized by a gradient-based one side sampling (GOSS) algorithm, is trained with a learning rate of 0.01, and adopts a multi-class log loss indicator for multi-target classification. The importance ranking of an abiotic indicator is determined according to a number (split) of critical decisions made by the abiotic indicator in a decision tree and an information gain.

The abiotic factors are input into the LightGBM model to obtain importance and importance ranking of each abiotic indicator among the abiotic factors.

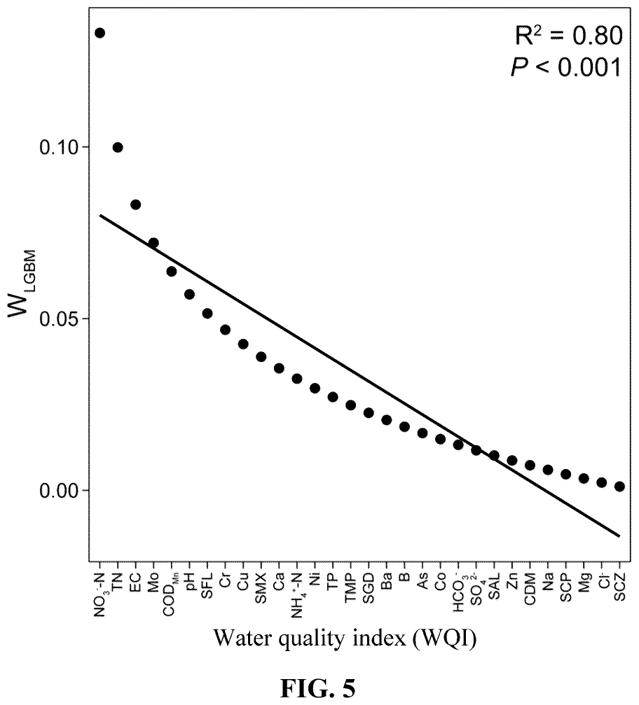

Based on the importance and importance ranking of each abiotic indicator among the abiotic factors, the machine learning-based abiotic factor weight matrix is determined by a rank order centroid method, as shown in FIG. 5.

$${W}_{LGBM} = [ \begin{matrix}\frac{1}{N} {\sum }_{RANK = 1}^{N} \frac{1}{RANK ( F [ i ] )} \\ \frac{1}{N} {\sum }_{RANK = 2}^{N} \frac{1}{RANK ( F [ i ] )} \\ ⋮ \\ \frac{1}{N} {\sum }_{RANK = N}^{N} \frac{1}{RANK ( F [ i ] )}\end{matrix} ]$$

where F[i] represents importance of an ith abiotic indicator among the abiotic factors, RANK(F[i]) represents importance ranking of the ith abiotic indicator among the abiotic factors, W_{LGBM} represents the machine learning-based abiotic factor weight matrix, and RANK represents importance ranking.

Step 105: An abiotic factor comprehensive weight matrix is determined according to the biotic-abiotic response relationship-based abiotic factor weight matrix and the machine learning-based abiotic factor weight matrix.

Based on a game theory, an optimal weight is determined by optimizing a weight coefficient in an equation to allow a minimum deviation between the biotic-abiotic response relationship-based abiotic factor weight matrix and the machine learning-based abiotic factor weight matrix.

A first weight coefficient and a second weight coefficient are determined according to the biotic-abiotic response relationship-based abiotic factor weight matrix and the machine learning-based abiotic factor weight matrix as follows:

$$[ \begin{matrix}{W}_{mic}^{T} {W}_{mic} & {W}_{LGBM}^{T} {W}_{mic} \\ {W}_{mic}^{T} {W}_{LGBM} & {W}_{LGBM}^{T} {W}_{LGBM}\end{matrix} ] [ \begin{matrix}{α}_{1} \\ {α}_{2}\end{matrix} ] = [ \begin{matrix}{W}_{mic}^{T} {W}_{mic} \\ {W}_{LGBM}^{T} {W}_{LGBM}\end{matrix} ] .$$

The abiotic factor comprehensive weight matrix obtained based on the game theory combines a biotic-abiotic factor response relationship and machine learning model training, which fully considers the response of biotic communities to WQIs and the influence of WQI concentrations on a water quality grade, avoids the subjectivity and uncertainty of expert grading, and reduces the one-sidedness of single physical and chemical concentrations for a result of a water quality assessment model.

A weight coefficient of the biotic-abiotic response relationship-based abiotic factor weight matrix and a weight coefficient of the machine learning-based abiotic factor weight matrix are determined based on the first weight coefficient and the second weight coefficient as follows:

$${α}_{1}^{*} = \frac{{α}_{1}}{{α}_{1} + {α}_{2}} \\ and \\ {α}_{2}^{*} = \frac{{α}_{2}}{{α}_{1} + {α}_{2}} .$$

The abiotic factor comprehensive weight matrix is determined according to the biotic-abiotic response relationship-based abiotic factor weight matrix, the machine learning-based abiotic factor weight matrix, the weight coefficient of the biotic-abiotic response relationship-based abiotic factor weight matrix, and the weight coefficient of the machine learning-based abiotic factor weight matrix as follows:

$$W = {α}_{1}^{*} {W}_{mic} + {α}_{2}^{*} {W}_{LGBM}$$

where

$${W}_{mic}^{T}$$

represents a transpose of the biotic-abiotic response relationship-based abiotic factor weight matrix,

$${W}_{LGBM}^{T}$$

represents a transpose of the machine learning-based abiotic factor weight matrix, α₁ represents the first weight coefficient, α₂ represents the second weight coefficient,

$${α}_{1}^{*}$$

represents the weight coefficient of the biotic-abiotic response relationship-based abiotic factor weight matrix,

$${α}_{2}^{*}$$

represents the weight coefficient of the machine learning-based abiotic factor weight matrix, and W represents the abiotic factor comprehensive weight matrix.

Step 106: The comprehensive water quality assessment of the water body to be tested is conducted based on the abiotic factor comprehensive weight matrix and the abiotic factors to determine a comprehensive water quality assessment result of the water body to be tested. The comprehensive water quality assessment result is provided to characterize a water quality safety status.

Each abiotic indicator among the abiotic factors is subjected to dimensionless value transformation, and a value range of each abiotic indicator among the abiotic factors is mapped to an interval [0,100] through linear interpolation.

Each abiotic indicator among the abiotic factors is mapped through the linear interpolation to obtain a factor index of each abiotic indicator among the abiotic factors.

$${SI}_{i} = ( {S}_{1} - {S}_{2} ) - \frac{( {S}_{1} \times {x}_{i} )}{( {x}_{2 , i} - {x}_{1 , i} )} , \\ {SI}_{i} = \frac{( {x}_{i} - {x}_{1 , i} )}{( {x}_{2 , i} - {x}_{1 , i} )} \times {S}_{1} , \\ and \\ {SI}_{i} = ( {S}_{1} - {S}_{2} ) - \frac{( {x}_{i} - {x}_{1 , i} )}{( {x}_{2 , i} - {x}_{1 , i} )} \times {S}_{1}$$

where SIᵢ represents a factor index calculated for an ith abiotic indicator among the abiotic factors; S₁ and S₂ represent range values corresponding to upper and lower limits of all WQIs (abiotic indicators) and are 100 and 0, respectively; X_{1,i} represents an allowed upper limit of an i th abiotic indicator among the abiotic factors; and X_{2,i} represents an allowed lower limit of the ith abiotic indicator among the abiotic factors. Factor indexes for WQIs other than pH are calculated according to

$${SI}_{i} = ( {S}_{1} - {S}_{2} ) - \frac{( {x}_{i} - {x}_{1 , i} )}{( {x}_{2 , i} - {x}_{1 , i} )} \times {S}_{1} .$$

A factor index for pH is calculated as follows: When 5.0≤pH<7.5, the factor index for pH is calculated according to

$${SI}_{i} = \frac{( {x}_{i} - {x}_{1 , i} )}{( {x}_{2 , i} - {x}_{1 , i} )} \times {S}_{1} .$$

When 8.5<pH≤9.0, the factor index for pH is calculated according to

$${SI}_{i} = ( {S}_{1} - {S}_{2} ) - \frac{( {x}_{i} - {x}_{1 , i} )}{( {x}_{2 , i} - {x}_{1 , i )}} \times {S}_{1} .$$

When 7.5≤pH≤8.5, the factor index for pH is 100. When pH<5.0 or pH>9.0, the factor index for pH is 0. Subsequently, S₁ and S₂ are calculated by an arcsine function arcsin( ), and corresponding calculation equations are as follows:

$${S}_{1}^{′} = arc sin ( \frac{{S}_{1}}{100} ) \\ and \\ {S}_{2}^{′} = arc sin ( \frac{{S}_{2}}{100} )$$

where

$${S}_{1}^{′} and {S}_{2}^{′}$$

represent S₁ and S₂ produced after calculation by the arcsine function, and are 1.571 and 0, respectively.

The comprehensive water quality assessment result of the water body to be tested is determined based on the factor index of each abiotic indicator among the abiotic factors and the abiotic factor comprehensive weight matrix as follows:

$$WQI = 100 {\sum }_{i = 1}^{N} {w}_{i} Sin ( {SI}_{i} )$$

where WQI represents the comprehensive water quality assessment result of the water body to be tested, Wᵢ represents a weight value of an ith abiotic indicator among the abiotic factors in the abiotic factor comprehensive weight matrix, Wᵢ represents a factor in W, and Sin(SIᵢ) represents a sine transform value of a factor index of the ith abiotic indicator among the abiotic factors.

According to the obtained value of WQI, a water quality at each sampling site is determined, and determination criteria are as follows: When the value of WQI is 90 to 100, it indicates that water is clean. When the value of WQI is 75 to 90, it indicates that water is lightly polluted. When the value of WQI is 50 to 75, it indicates that water is moderately polluted. When the value of WQI is 25 to 50, it indicates that water is heavily polluted. When the value of WQI is 25 to 0, it indicates that water is seriously polluted and is black and odorous.

With a specified river as an example, a water quality is subjected to comprehensive assessment by integrating biotic and abiotic factors, including the following steps:
(1) According to characteristics of the river, 24 monitoring sections are set, and a water sample is collected from each section.
(2) Abiotic factors are determined. Physical and chemical property indicators such as pH, electrical conductivity (EC, us/cm), bicarbonates (HCO₃, mg/L), a permanganate index (COD_{Mn}, mg/L), total nitrogen (TN, mg/L), nitrate nitrogen (NO₃⁻—N, mg/L), ammonia nitrogen (NH₄⁺—N), TP (mg/L), chlorides (Cl⁻, mg/L), and sulfates (SO₄²⁻, mg/L) in a water sample are determined. Metal indicators such as sodium (Na, mg/L), magnesium (Mg, mg/L), calcium (Ca, mg/L), boron (B, μg/L), chromium (Cr, μg/L), cobalt (Co, μg/L), nickel (Ni, μg/L), copper (Cu, μg/L), zinc (Zn, μg/L), barium (Ba, μg/L), arsenic (As, μg/L), and molybdenum (Mo, μg/L) in a water sample are determined. Antibiotic indicators such as sulfonamides and tetracyclines in a water sample are determined.
(3) Biotic factors are determined. DNA is extracted from each sample. 16S rRNA primers (341F and 518R) are adopted for the detection of bacteria. 349F and 806R primers are adopted for the detection of archaea. Universal primers for eukaryotes (1380F and 1510R) are adopted for the detection of fungi. 23S rRNA primers (A23SrVF1 and A23SrVF2) are adopted for the detection of algae. Primers (COlintF and HCO2198) corresponding to the mitochondrial gene COI are adopted for the detection of zoobenthos. Fish 12S rRNA primers (12SV5F and 12SV5R) are adopted for the detection of fish. The obtained high-throughput sequencing data is subjected to quality control and filtration, and sequences are clustered according to a nucleotide similarity of 97% to obtain OTU representative sequences. OTU representative sequences with a relative abundance of higher than 0.05% and a detection frequency of higher than 20% in all samples are used for downstream taxonomic analysis.
(4) The OTU representative sequences are subjected to taxonomic annotation by an RDP classifier Bayesian algorithm or a BLAST alignment approach. Based on abundance and taxonomic annotation results of the OTU representative sequences, diversities and relative species abundances of the biotic communities at different trophic levels are calculated. The co-existence relationship network of the biotic communities is constructed, and network topology properties of the biotic communities are assessed. Three types of biotic factors are set: 1) Microbial a diversities: ACE, Chao, Shannon, and Simpson indexes of bacterial, archaeal, fungal, algal, zoobenthic, and fish communities. 2) Microbial network indicators: At least one of a node number, an edge number, a network degree, assortativity, an edge density, an average path length, betweenness centrality, degree centralization, network transitivity, a network diameter, modularity, and vulnerability. 3) Microbial species composition: Relative abundances of bacterial, archaeal, fungal, algal, zoobenthic, and fish communities at each classification level.
(5) Spearman correlation between each abiotic indicator among the abiotic factors and each biotic indicator of the biotic communities at different trophic levels in the biotic factor indicator library is calculated, and a significance P value is obtained based on a correlation test, so as to obtain a biotic-abiotic response relationship-based abiotic factor weight matrix.
(6) According to national and international standards such as Environmental Quality Standards for Surface Water (GB3838-2002), a water quality category at each sampling site is determined (subject to the worst indicator category). A machine learning-based abiotic factor weight matrix is calculated according to the LightGBM machine learning algorithm.
(7) Weight coefficients of the biotic-abiotic response relationship-based abiotic factor weight matrix and the machine learning-based abiotic factor weight matrix are calculated. Calculated weight values of WQIs (abiotic indicators) are shown in Table 1. There are great differences among WQIs in terms of the importance for the water quality grade determination based on a concentration threshold and a role in a microbial response. For example, the LightGBM algorithm shows that TN and NO₃⁻—N are main factors causing a change of a water quality grade, but are less associated with diversities, species abundances, and network structures of biotic communities as non-restrictive nutrients in water. The biotic communities often exhibit sensitive dynamics related to a plurality of WQIs, and environmental responses and disturbance intensities of the biotic communities often imply the fluctuation in a water quality or the intervention of a plurality of external environmental factors. The microbial community analysis and the machine learning algorithm comprehensively consider the physical and chemical concentrations and biotic impacts of different WQIs, which is conducive to providing a more comprehensive and accurate water quality assessment system than the traditional WQI model.

TABLE 1
Calculation of weights of WQIs

| | Spearman | | Importance ranking | | |
|---|---|---|---|---|---|
| WQI | correlation | W_{mic} | of LightGBM | W_{LGBM} | W |
| NO₃⁻—N | 0.0690 | 0.0043 | 1 | 0.1332 | 0.0809 |
| EC | 0.8276 | 0.0521 | 3 | 0.0832 | 0.0705 |
| TN | 0.0690 | 0.0043 | 2 | 0.0998 | 0.0611 |
| COD_{Mn} | 0.7241 | 0.0456 | 5 | 0.0637 | 0.0563 |
| Mo | 0.2759 | 0.0174 | 4 | 0.0721 | 0.0499 |
| Ni | 0.9310 | 0.0586 | 13 | 0.0297 | 0.0414 |
| Ca | 0.7241 | 0.0456 | 11 | 0.0355 | 0.0396 |
| SFL | 0.3448 | 0.0217 | 7 | 0.0515 | 0.0394 |
| Cr | 0.4483 | 0.0282 | 8 | 0.0467 | 0.0392 |
| pH | 0.2069 | 0.0130 | 6 | 0.0571 | 0.0392 |
| Cu | 0.3793 | 0.0239 | 9 | 0.0426 | 0.0350 |
| TP | 0.6552 | 0.0412 | 14 | 0.0272 | 0.0329 |
| TMP | 0.6897 | 0.0434 | 15 | 0.0248 | 0.0323 |
| SMX | 0.3448 | 0.0217 | 10 | 0.0389 | 0.0319 |
| SAL | 1.0000 | 0.0629 | 23 | 0.0101 | 0.0316 |
| HCO₃⁻ | 0.8966 | 0.0564 | 21 | 0.0132 | 0.0308 |
| Ba | 0.6207 | 0.0390 | 17 | 0.0205 | 0.0280 |
| SGD | 0.5517 | 0.0347 | 16 | 0.0226 | 0.0275 |
| B | 0.6207 | 0.0390 | 18 | 0.0185 | 0.0268 |
| Co | 0.6207 | 0.0390 | 20 | 0.0149 | 0.0247 |
| Na | 0.8276 | 0.0521 | 26 | 0.0060 | 0.0247 |
| Mg | 0.8621 | 0.0542 | 28 | 0.0035 | 0.0241 |
| NH₄⁺—N | 0.1724 | 0.0108 | 12 | 0.0325 | 0.0237 |
| CDM | 0.7586 | 0.0477 | 25 | 0.0073 | 0.0237 |
| SO₄²⁻ | 0.6552 | 0.0412 | 22 | 0.0117 | 0.0237 |
| Cl | 0.8276 | 0.0521 | 29 | 0.0023 | 0.0225 |
| Zn | 0.6207 | 0.0390 | 24 | 0.0087 | 0.0210 |
| As | 0.0345 | 0.0022 | 19 | 0.0167 | 0.0108 |
| SCP | 0.1379 | 0.0087 | 27 | 0.0047 | 0.0063 |
| SCZ | 0.0000 | 0.0000 | 30 | 0.0011 | 0.0007 |

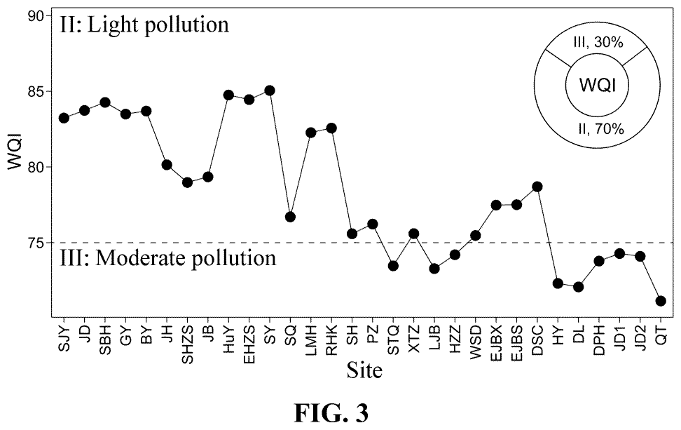

(8) Each abiotic indicator among the abiotic factors is subjected to dimensionless value transformation, and the linear interpolation is optimized by the arcsine function.
(9) Water quality grades matching different WQI value ranges are defined to assess a water quality at each sampling site of the river. A WQI value tends to decrease along the river overall, and an average value is 78.3. The model shows that water at these sampling sites is classified as grade III water and has a pollution grade of “moderate pollution”, and water at the remaining sampling sites has a pollution grade of “light pollution”, as shown in FIG. 3. A WQI value and a corresponding water quality grade at each sampling site are shown in Table 2.

TABLE 2
A WQI value and a water quality grade at each sampling site

| No. | WQI value | Water quality grade |
|---|---|---|
| 1 | 83.24 | Light pollution |
| 2 | 83.74 | Light pollution |
| 3 | 84.27 | Light pollution |
| 4 | 83.50 | Light pollution |
| 5 | 83.70 | Light pollution |
| 6 | 80.15 | Light pollution |
| 7 | 78.98 | Light pollution |
| 8 | 79.35 | Light pollution |
| 9 | 84.76 | Light pollution |
| 10 | 84.46 | Light pollution |
| 11 | 85.06 | Light pollution |
| 12 | 76.70 | Light pollution |
| 13 | 82.27 | Light pollution |
| 14 | 82.57 | Light pollution |
| 15 | 75.59 | Light pollution |
| 16 | 76.23 | Light pollution |
| 17 | 73.47 | Moderate pollution |
| 18 | 75.60 | Light pollution |
| 19 | 73.28 | Moderate pollution |
| 20 | 74.20 | Moderate pollution |
| 21 | 75.48 | Light pollution |
| 22 | 77.48 | Light pollution |
| 23 | 77.51 | Light pollution |
| 24 | 78.70 | Light pollution |
| 25 | 72.31 | Moderate pollution |
| 26 | 72.08 | Moderate pollution |
| 27 | 73.79 | Moderate pollution |
| 28 | 74.28 | Moderate pollution |
| 29 | 74.10 | Moderate pollution |
| 30 | 71.15 | Moderate pollution |

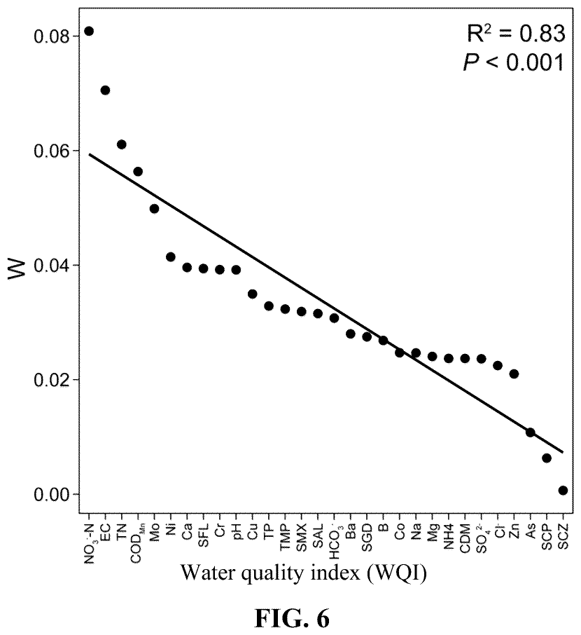

(10) Result reliability analysis: The W_{mic}, W_{LGBM}, and W_{weight} distributions are analyzed through ranking fitting, as shown in FIG. 6. The fitting of weights in an order from low to high can well explain a structure of weights. The higher the fitting, the more stable the structure of weights. A goodness-of-fit of the biotic-abiotic response relationship-based abiotic factor weight matrix is 0.97, and is significantly higher than a weight value obtained by the machine learning-based abiotic factor weight matrix, reflecting prominent model adaptability. A goodness-of-fit of the abiotic factor comprehensive weight matrix is 0.83, and is statistically significant, indicating that a WQI weight set calculated based on microbial community analysis and a machine learning algorithm is reliable.

The present disclosure has the following technical effects:
1. The present disclosure addresses the need for comprehensive water quality assessment of rivers, lakes, and reservoirs by integrating multiple biotic and abiotic factors. It emphasizes practicality and potential for widespread application. Through the establishment of a biotic-abiotic factor response relationship and training with machine learning models, the present disclosure aims to derive relative importance information for indicators that is both scientifically sound and practically applicable. This approach ensures the objectivity and utility of indicator weights, thereby enhancing the effectiveness of assessing and managing environmental conditions in aquatic ecosystems.
2. Based on all biotic and abiotic factors, the present disclosure integrates environmental factors such as basic physical and chemical properties, heavy metals, and emerging contaminants into a comprehensive assessment framework for assessing the safety status of water sources. This approach involves quantitative analysis and decision-making techniques to avoid the uncertainties associated with subjective judgments. By quantitatively evaluating multiple influencing factors from various sources, the present disclosure provides technical support aimed at ensuring water quality safety in rivers, lakes, and reservoirs. This method enhances the reliability and objectivity of assessing environmental conditions, thereby facilitating more effective management and protection of aquatic ecosystems.

Example 2: A computer system is provided, including: a memory, a processor, and a computer program stored in the memory and runnable on the processor. The processor is configured to execute the computer program to implement the steps of the method for comprehensive water quality assessment by integrating biotic and abiotic factors in Example 1.

Example 3: A computer-readable storage medium is provided. A computer program is stored in the computer-readable storage medium, and when executed by a processor, the computer program implements the steps of the method for comprehensive water quality assessment by integrating biotic and abiotic factors in Example 1.

Example 4: A computer program product is provided, including a computer program. When executed by a processor, the computer program implements the steps of the method for comprehensive water quality assessment by integrating biotic and abiotic factors in Example 1.

Example 5: A computer apparatus is provided. The computer apparatus may be a database. The computer apparatus includes a processor, a memory, an input/output (I/O) interface, and a communication interface. The processor, the memory, and the I/O interface are connected through a system bus, and the communication interface is connected to the system bus through the I/O interface. The processor of the computer apparatus is configured to provide computing and control capabilities. The memory of the computer apparatus includes a non-volatile storage medium and an internal memory. The non-volatile storage medium stores an operating system, a computer program, and a database. The internal memory provides an environment for operations of the operating system and the computer program in the non-volatile storage medium. The database of the computer apparatus is configured to store pending transactions. The I/O interface of the computer apparatus is configured to exchange information between the processor and an external apparatus. The communication interface of the computer apparatus is configured to communicate with an external terminal through a network. When executed by the processor, the computer program implements the method for comprehensive water quality assessment by integrating biotic and abiotic factors in Example 1.

It should be noted that the object information (including, but not limited to, object apparatus information, object personal information, or the like) and data (including, but not limited to, data for analysis, stored data, displayed data, or the like) involved in the present disclosure all are information and data authorized by an object or fully authorized by all parties, and the acquisition, use, and processing of relevant data need to comply with the relevant laws, regulations, and standards of relevant countries and regions.

Those of ordinary skill in the art may understand that all or some of the procedures in the method of the above embodiment may be implemented by a computer program commanding related hardware. The computer program may be stored in a non-volatile computer-readable storage medium. When the computer program is executed, the procedures in the embodiment of the above method may be implemented. Any reference to a memory, a database, or other media used in the embodiments of the present disclosure may include at least one of non-volatile and volatile memories. Non-volatile memories may include a read-only memory (ROM), a magnetic tape, a floppy disk, a flash memory, an optical memory, a high-density embedded non-volatile memory, a resistive random access memory (ReRAM), a magnetoresistive random access memory (MRAM), a ferroelectric random access memory (FRAM), a phase change memory (PCM), a graphene memory, or the like. Volatile memories may include a random access memory (RAM) or an external cache memory. As an illustration rather than a limitation, the RAM may be in various forms, such as a static random access memory (SRAM) or a dynamic random access memory (DRAM). The database involved in each embodiment provided by the present disclosure may include at least one of a relational database and a non-relational database. The non-relational database can include a block chain-based distributed database, but is not limited thereto. The processor involved in each embodiment provided by the present disclosure may be a general-purpose processor, a central processor, a graphic processor, a digital signal processor, a programmable logic device, or a quantum computing-based data processing logic device, but is not limited thereto.

The technical characteristics of the above embodiments can be arbitrarily combined. For brevity of description, not all possible combinations of the technical characteristics of the above embodiments are described. However, these combinations of the technical characteristics should be construed as falling within the scope defined by the specification as long as there is no contradiction among the combinations.

Specific examples are used herein to explain the principles and implementations of the present disclosure. The description of the examples is merely intended to help understand the method of the present disclosure and its core ideas. In addition, those of ordinary skill in the art can make various modifications to the specific implementations and application scope in accordance with the teachings of the present disclosure. In conclusion, the content of the present specification shall not be construed as a limitation to the present disclosure.

What is claimed is:

1. A method for comprehensive water quality assessment by integrating biotic and abiotic factors, comprising:
acquiring abiotic factors of a water body to be tested, wherein the water body to be tested comprises a river, a lake, and a reservoir; the abiotic factors comprise different abiotic indicators; and the different abiotic indicators are pH, dissolved oxygen, total dissolved solids, a permanganate index, ammonia nitrogen, nitrate nitrogen, total nitrogen, total phosphorus (TP), chlorides, sulfates, Na, Fe, Ca, Mg, Cu, Zn, Cr, As, Mo, antibiotics, or perfluorinated compounds;
constructing a biotic factor indicator library by an environmental DNA technology, wherein the biotic factor indicator library comprises different biotic indicators of biotic communities at different trophic levels; the biotic communities at different trophic levels are bacterial communities, archaeal communities, fungal communities, algal communities, zoobenthic communities, or fish communities; and the different biotic indicators are diversity indexes, relative abundances at each classification level, or co-occurrence network topology properties;
determining a biotic-abiotic response relationship-based abiotic factor weight matrix using the abiotic factors and the biotic factor indicator library;
acquiring a machine learning-based abiotic factor weight matrix using the abiotic factors and a LightGBM model, wherein the LightGBM model is configured to determine importance of each abiotic indicator among the abiotic factors relative to water quality;
determining an abiotic factor comprehensive weight matrix according to the biotic-abiotic response relationship-based abiotic factor weight matrix and the machine learning-based abiotic factor weight matrix; and
conducting the comprehensive water quality assessment of the water body to be tested based on the abiotic factor comprehensive weight matrix and the abiotic factors to determine a comprehensive water quality assessment result of the water body to be tested, wherein the comprehensive water quality assessment result is provided to characterize a water quality safety status.

2. The method for comprehensive water quality assessment by integrating biotic and abiotic factors according to claim 1, wherein the constructing a biotic factor indicator library by an environmental DNA technology specifically comprises:
acquiring high-throughput sequencing data of the biotic communities at different trophic levels in the water body to be tested by the environmental DNA technology;
subjecting the high-throughput sequencing data of the biotic communities at different trophic levels to quality control and filtration to obtain processed high-throughput sequencing data of the biotic communities at different trophic levels;
clustering the processed high-throughput sequencing data of the biotic communities at different trophic levels to obtain operational taxonomic unit (OTU) representative sequences; and
subjecting the OTU representative sequences to taxonomic annotation by a taxonomic approach to calculate diversity indexes, relative abundances at each classification level, or co-occurrence network topology properties of the biotic communities at different trophic levels, wherein the diversity indexes comprise ACE, Chao, Shannon, and Simpson indexes; and the co-occurrence network topology properties comprise at least one of a node number, an edge number, a network degree, assortativity, an edge density, an average path length, betweenness centrality, degree centralization, network transitivity, a network diameter, modularity, and vulnerability.

3. The method for comprehensive water quality assessment by integrating biotic and abiotic factors according to claim 2, wherein the taxonomic approach is any one of a ribosomal database project (RDP) classifier Bayesian algorithm and a basic local alignment search tool (BLAST) alignment approach.

4. The method for comprehensive water quality assessment by integrating biotic and abiotic factors according to claim 1, wherein the determining a biotic-abiotic response relationship-based abiotic factor weight matrix using the abiotic factors and the biotic factor indicator library specifically comprises:
calculating Spearman correlation between each abiotic indicator among the abiotic factors and each biotic indicator of the biotic communities at different trophic levels in the biotic factor indicator library;
testing the Spearman correlation between each abiotic indicator among the abiotic factors and each biotic indicator of the biotic communities at different trophic levels in the biotic factor indicator library to obtain a significance P value between each abiotic indicator among the abiotic factors and each biotic indicator of the biotic communities at different trophic levels in the biotic factor indicator library;
constructing a significance P value matrix based on the significance P value between each abiotic indicator among the abiotic factors and each biotic indicator of the biotic communities at different trophic levels in the biotic factor indicator library;
defining a significance P value in the significance P value matrix that satisfies a preset condition as 1, and defining a significance P value in the significance P value matrix that does not satisfy the preset condition as 0, so as to obtain a 0-1 correlation matrix; and
standardizing and normalizing the 0-1 correlation matrix to obtain the biotic-abiotic response relationship-based abiotic factor weight matrix.

5. The method for comprehensive water quality assessment by integrating biotic and abiotic factors according to claim 1, wherein the acquiring a machine learning-based abiotic factor weight matrix using the abiotic factors and a LightGBM model specifically comprises:
inputting the abiotic factors into the LightGBM model to obtain importance and importance ranking of each abiotic indicator among the abiotic factors; and
based on the importance and importance ranking of each abiotic indicator among the abiotic factors, determining the machine learning-based abiotic factor weight matrix by a rank order centroid method:
$${W}_{LGBM} = [ \begin{matrix}\frac{1}{N} {\sum }_{RANK = 1}^{N} \frac{1}{RANK ( F [ i ] )} \\ \frac{1}{N} {\sum }_{RANK = 2}^{N} \frac{1}{RANK ( F [ i ] )} \\ ⋮ \\ \frac{1}{N} {\sum }_{RANK = N}^{N} \frac{1}{RANK ( F [ i ] )}\end{matrix} ] ,$$
wherein N represents a number of abiotic indicators among the abiotic factors; F[i] represents importance of an i th abiotic indicator among the abiotic factors; RANK(F[i]) represents importance ranking of the ith abiotic indicator among the abiotic factors; and W_{LGBM} represents the machine learning-based abiotic factor weight matrix.

6. The method for comprehensive water quality assessment by integrating biotic and abiotic factors according to claim 1, wherein the determining an abiotic factor comprehensive weight matrix according to the biotic-abiotic response relationship-based abiotic factor weight matrix and the machine learning-based abiotic factor weight matrix specifically comprises:
determining a first weight coefficient and a second weight coefficient with a game theory according to the biotic-abiotic response relationship-based abiotic factor weight matrix and the machine learning-based abiotic factor weight matrix:
$$[ \begin{matrix}{W}_{mic}^{T} {W}_{mic} & {W}_{LGBM}^{T} {W}_{mic} \\ {W}_{mic}^{T} {W}_{LGBM} & {W}_{LGBM}^{T} {W}_{LGBM}\end{matrix} ] [ \begin{matrix}{α}_{1} \\ {α}_{2}\end{matrix} ] = [ \begin{matrix}{W}_{mic}^{T} {W}_{mic} \\ {W}_{LGBM}^{T} {W}_{LGBM}\end{matrix} ] ;$$
determining a weight coefficient of the biotic-abiotic response relationship-based abiotic factor weight matrix and a weight coefficient of the machine learning-based abiotic factor weight matrix based on the first weight coefficient and the second weight coefficient:
$${α}_{1}^{*} = \frac{{α}_{1}}{{α}_{1} + {α}_{2}} \\ and \\ {α}_{2}^{*} = \frac{{α}_{2}}{{α}_{1} + {α}_{2}} ;$$
and
determining the abiotic factor comprehensive weight matrix according to the biotic-abiotic response relationship-based abiotic factor weight matrix, the machine learning-based abiotic factor weight matrix, the weight coefficient of the biotic-abiotic response relationship-based abiotic factor weight matrix, and the weight coefficient of the machine learning-based abiotic factor weight matrix:
$$W = {α}_{1}^{*} {W}_{mic} + {α}_{2}^{*} {W}_{LGBM} ,$$
wherein
$${W}_{mic}^{T}$$
represents a transpose of the biotic-abiotic response relationship-based abiotic factor weight matrix, W_{mic} represents the biotic-abiotic response relationship-based abiotic factor weight matrix, W_{LGBM} represents the machine learning-based abiotic factor weight matrix,
$${W}_{LGBM}^{T}$$
represents a transpose of the machine learning-based abiotic factor weight matrix, α₁ represents the first weight coefficient, α₂ represents the second weight coefficient,
$${α}_{1}^{*}$$
represents the weight coefficient of the biotic-abiotic response relationship-based abiotic factor weight matrix,
$${α}_{2}^{*}$$
represents the weight coefficient of the machine learning-based abiotic factor weight matrix, and/represents the abiotic factor comprehensive weight matrix.

7. The method for comprehensive water quality assessment by integrating biotic and abiotic factors according to claim 1, wherein conducting the comprehensive water quality assessment of the water body to be tested based on the abiotic factor comprehensive weight matrix and the abiotic factors to determine a comprehensive water quality assessment result of the water body to be tested specifically comprises:
mapping each abiotic indicator among the abiotic factors through linear interpolation to obtain a factor index of each abiotic indicator among the abiotic factors; and
determining the comprehensive water quality assessment result of the water body to be tested based on the factor index of each abiotic indicator among the abiotic factors and the abiotic factor comprehensive weight matrix:
$$WQI = 100 {\sum }_{i = 1}^{N} {w}_{i} Sin ( {SI}_{i} ) ,$$
wherein WQI represents the comprehensive water quality assessment result of the water body to be tested, Wᵢ represents a weight value of an ith abiotic indicator among the abiotic factors in the abiotic factor comprehensive weight matrix, SIᵢ represents a factor index of the ith abiotic indicator among the abiotic factors, N represents a number of abiotic indicators among the abiotic factors, and Sin(SIᵢ) represents a sine transform value of the factor index of the ith abiotic indicator among the abiotic factors.

8. A computer system, comprising: a memory, a processor, and a computer program stored in the memory and runnable on the processor, wherein the processor is configured to execute the computer program to implement the steps of the method for comprehensive water quality assessment by integrating biotic and abiotic factors according to claim 1.

9. The computer system according to claim 8, wherein the constructing a biotic factor indicator library by an environmental DNA technology specifically comprises:
acquiring high-throughput sequencing data of the biotic communities at different trophic levels in the water body to be tested by the environmental DNA technology;
subjecting the high-throughput sequencing data of the biotic communities at different trophic levels to quality control and filtration to obtain processed high-throughput sequencing data of the biotic communities at different trophic levels;
clustering the processed high-throughput sequencing data of the biotic communities at different trophic levels to obtain operational taxonomic unit (OTU) representative sequences; and
subjecting the OTU representative sequences to taxonomic annotation by a taxonomic approach to calculate diversity indexes, relative abundances at each classification level, or co-occurrence network topology properties of the biotic communities at different trophic levels, wherein the diversity indexes comprise ACE, Chao, Shannon, and Simpson indexes; and the co-occurrence network topology properties comprise at least one of a node number, an edge number, a network degree, assortativity, an edge density, an average path length, betweenness centrality, degree centralization, network transitivity, a network diameter, modularity, and vulnerability.

10. The computer system according to claim 9, wherein the taxonomic approach is any one of a ribosomal database project (RDP) classifier Bayesian algorithm and a basic local alignment search tool (BLAST) alignment approach.

11. The computer system according to claim 8, wherein the determining a biotic-abiotic response relationship-based abiotic factor weight matrix using the abiotic factors and the biotic factor indicator library specifically comprises:
calculating Spearman correlation between each abiotic indicator among the abiotic factors and each biotic indicator of the biotic communities at different trophic levels in the biotic factor indicator library;
testing the Spearman correlation between each abiotic indicator among the abiotic factors and each biotic indicator of the biotic communities at different trophic levels in the biotic factor indicator library to obtain a significance P value between each abiotic indicator among the abiotic factors and each biotic indicator of the biotic communities at different trophic levels in the biotic factor indicator library;
constructing a significance P value matrix based on the significance P value between each abiotic indicator among the abiotic factors and each biotic indicator of the biotic communities at different trophic levels in the biotic factor indicator library;
defining a significance P value in the significance P value matrix that satisfies a preset condition as 1, and defining a significance P value in the significance P value matrix that does not satisfy the preset condition as 0, so as to obtain a 0-1 correlation matrix; and
standardizing and normalizing the 0-1 correlation matrix to obtain the biotic-abiotic response relationship-based abiotic factor weight matrix.

12. The computer system according to claim 8, wherein the acquiring a machine learning-based abiotic factor weight matrix using the abiotic factors and a LightGBM model specifically comprises:
inputting the abiotic factors into the LightGBM model to obtain importance and importance ranking of each abiotic indicator among the abiotic factors; and
based on the importance and importance ranking of each abiotic indicator among the abiotic factors, determining the machine learning-based abiotic factor weight matrix by a rank order centroid method:
$${W}_{LGBM} = [ \begin{matrix}\frac{1}{N} {\sum }_{RANK = 1}^{N} \frac{1}{RANK ( F [ i ] )} \\ \frac{1}{N} {\sum }_{RANK = 2}^{N} \frac{1}{RANK ( F [ i ] )} \\ ⋮ \\ \frac{1}{N} {\sum }_{RANK = N}^{N} \frac{1}{RANK ( F [ i ] )}\end{matrix} ] ,$$
wherein N represents a number of abiotic indicators among the abiotic factors; F[i] represents importance of an i th abiotic indicator among the abiotic factors; RANK(F[i]) represents importance ranking of the ith abiotic indicator among the abiotic factors; and W_{LGBM} represents the machine learning-based abiotic factor weight matrix.

13. The computer system according to claim 8, wherein the determining an abiotic factor comprehensive weight matrix according to the biotic-abiotic response relationship-based abiotic factor weight matrix and the machine learning-based abiotic factor weight matrix specifically comprises:
determining a first weight coefficient and a second weight coefficient with a game theory according to the biotic-abiotic response relationship-based abiotic factor weight matrix and the machine learning-based abiotic factor weight matrix:
$$[ \begin{matrix}{W}_{mic}^{T} {W}_{mic} & {W}_{LGBM}^{T} {W}_{mic} \\ {W}_{mic}^{T} {W}_{LGBM} & {W}_{LGBM}^{T} {W}_{LGBM}\end{matrix} ] [ \begin{matrix}{α}_{1} \\ {α}_{2}\end{matrix} ] = [ \begin{matrix}{W}_{mic}^{T} {W}_{mic} \\ {W}_{LGBM}^{T} {W}_{LGBM}\end{matrix} ] ;$$
determining a weight coefficient of the biotic-abiotic response relationship-based abiotic factor weight matrix and a weight coefficient of the machine learning-based abiotic factor weight matrix based on the first weight coefficient and the second weight coefficient:
$${α}_{1}^{*} = \frac{{α}_{1}}{{α}_{1} + {α}_{2}} and$$
$${α}_{2}^{*} = \frac{{α}_{2}}{{α}_{1} + {α}_{2}} ;$$
and
determining the abiotic factor comprehensive weight matrix according to the biotic-abiotic response relationship-based abiotic factor weight matrix, the machine learning-based abiotic factor weight matrix, the weight coefficient of the biotic-abiotic response relationship-based abiotic factor weight matrix, and the weight coefficient of the machine learning-based abiotic factor weight matrix:
$$W = {α}_{1}^{*} {W}_{mic} + {α}_{2}^{*} {W}_{LGBM} ,$$
wherein
$${W}_{mic}^{T}$$
represents a transpose of the biotic-abiotic response relationship-based abiotic factor weight matrix, W_{mic} represents the biotic-abiotic response relationship-based abiotic factor weight matrix, W_{LGBM} represents the machine learning-based abiotic factor weight matrix,
$${W}_{LGBM}^{T}$$
represents a transpose of the machine learning-based abiotic factor weight matrix, α₁ represents the first weight coefficient, α₂ represents the second weight coefficient,
$${α}_{1}^{*}$$
represents the weight coefficient of the biotic-abiotic response relationship-based abiotic factor weight matrix,
$${α}_{2}^{*}$$
represents the weight coefficient of the machine learning-based abiotic factor weight matrix, and W represents the abiotic factor comprehensive weight matrix.

14. The computer system according to claim 8, wherein conducting the comprehensive water quality assessment of the water body to be tested based on the abiotic factor comprehensive weight matrix and the abiotic factors to determine a comprehensive water quality assessment result of the water body to be tested specifically comprises:
mapping each abiotic indicator among the abiotic factors through linear interpolation to obtain a factor index of each abiotic indicator among the abiotic factors; and
determining the comprehensive water quality assessment result of the water body to be tested based on the factor index of each abiotic indicator among the abiotic factors and the abiotic factor comprehensive weight matrix:
$$WQI = 100 {\sum }_{i = 1}^{N} {w}_{i} Sin ( {SI}_{i} ) ,$$
wherein WQI represents the comprehensive water quality assessment result of the water body to be tested, Wᵢ represents a weight value of an ith abiotic indicator among the abiotic factors in the abiotic factor comprehensive weight matrix, SIᵢ represents a factor index of the ith abiotic indicator among the abiotic factors, N represents a number of abiotic indicators among the abiotic factors, and Sin(SIᵢ) represents a sine transform value of the factor index of the ith abiotic indicator among the abiotic factors.
